(19) United States
(12) Patent Application Publication
(10) Pub. No.: US 2024/0410889 A1
(43) Pub. Date: Dec. 12, 2024

(54) CHIP-BASED CARTRIDGE FOR BIOLOGICAL DETECTION AND USES THEREOF

(71) Applicants: ERICK WANG, Hsinchu City (TW); AGRITALK TECHNOLOGY INC., Zhubei City (TW)

(72) Inventors: WEN-LIANG CHEN, Hsinchu (TW); LUNG-CHIEH CHEN, Hsinchu (TW); CHIN-HSIEN HSIAO, Zhubei City (TW); JEN-CHIEH WEI, Taipei City (TW); SZ-HAU CHEN, Taipei City (TW); YOU-SYUAN GAO, Taipei City (TW); ERICK WANG, Hsinchu City (TW)

(21) Appl. No.: 18/702,334

(22) PCT Filed: Oct. 18, 2021
(86) PCT No.: PCT/US21/55339

§ 371 Date: Apr. 18, 2024

(51) Int. Cl. G01N 33/569; B01L 3/00; G01N 33/543

(52) U.S. Cl. CPC G01N 33/56983; B01L 3/502761; G01N 33/54386; B01L 2200/04; B01L 2200/0647; B01L 2200/16; G01N 2333/165; G01N 2469/10

(57) ABSTRACT

The present invention provides a chip-based cartridge for biological detection and a detection method thereof, comprising a body formed by a flow guiding layer stacking with a substrate. The substrate has a reaction area positioned on part of the electrodes, and the reaction area is coated with a receptor for binding the detection target; The flow guiding layer has an opening aligned with the reaction area, the receptor is exposed at the opening corresponding to the body, and a microfluidic channel is formed between the flow guiding layer and the substrate; The sample is directly dropped from the opening to the reaction area to react with the receptor. After the reaction time, wash buffer is added to the sample in the reaction area. Then, the detection buffer is dropped into the reaction area so as to complete the sample detection without external driving force while improving the detection efficiency.

### CROSS-REFERENCE TO RELATED APPLICATION

This application is the US national phase of international application No. PCT/US21/55339 filed on Oct. 18, 2021. The contents of the above applications are incorporated herein by reference in their entirety.

### BACKGROUND OF THE INVENTION

### 1. Field of the Invention

The present invention provides a detection device, in particular a chip-based cartridge for biological detection and the detection method thereof.

### 2. Description of the Related Art

### BACKGROUND

A conventional biochip detection device is coated with a receptor for binding the detection target in the reaction area where the reaction area is configured within the detection device without being exposed. During detection, the operator takes an available amount of sample (usually biological body fluid such as blood) and drops it at the inlet of the reaction area, and then forwards the sample to the reaction area by applying force (such as pressurized gas) provided by an external power source. The sample then comes into contact with the receptor and the resulting signal is recorded, so as to obtain the analysis results. However, sample integrity may be compromised when such outside force is applied, resulting in inaccurate analysis.

Another conventional design for detection is a paper-based test strip. For this method, the operator drops the sample at the inlet of the reaction area, the sample liquid is drained into the reaction area via the capillary action, and detected after contacting the receptor to obtain the analysis results. However, it takes time for the sample to be drained into the reaction area, and the reaction cannot proceed until the sample reaches the reaction area, which results in lengthy detection time.

### BRIEF SUMMARY OF THE INVENTION

The proposed chip-based cartridge for biological detection and the detection method thereof was developed in order to complete sample detection efficiently and without external driving force.

Accordingly, a first aspect of the present invention provides a chip-based cartridge for biological detection comprising a body. The body comprises a substrate and a flow guiding layer (layer that directs fluid flow). The substrate consists of electrodes and a reaction area positioned on the part of the electrodes, where a receptor is coated on the reaction area to bind a detection target; the flow guiding layer is stacked with the substrate. The flow guiding layer is provided with an opening aligned with the reaction area and the receptor is exposed at the opening corresponding to the body such that a sample can be dropped directly to the reaction area and perform a reaction with the receptor. Moreover, a microfluidic channel formed between the flow guiding layer and the substrate is configured to connect the reaction area. After the reaction, wash buffer is added, and both sample and wash buffer are then drawn into the microfluidic channel and discharged.

In one embodiment, there is provided a chip-based cartridge where the flow guiding layer comprises a capillary hydrophilic layer and a non-absorbent hydrophobic layer, the hydrophobic layer is positioned between the substrate and the hydrophilic layer, the opening penetrates through the hydrophilic layer and the hydrophobic layer to the reaction area, and the hydrophobic layer has a space to form the microfluidic channel between the substrate and the hydrophilic layer.

In one embodiment, there is provided a chip-based cartridge where the opening is hexagonal and has a reaction section of uniform width as well as a tapered buffering section. The buffering section has a wide end and a narrow end; the wide end is arranged at one end of the reaction section, and the narrow end is connected to the space of the hydrophobic layer to connect to the microfluidic channel.

In one embodiment, there is provided a chip-based cartridge where the chip-based cartridge further comprises an adsorption part, the hydrophilic layer has a water outlet at one end of the microfluidic channel away from the reaction area, and the adsorption part is positioned on the water outlet configured to the hydrophilic layer.

In one embodiment, there is provided a chip-based cartridge where the chip-based cartridge further comprises a housing, the body and the adsorption part are covered and positioned by the housing, the housing has a window passing through the opening to the reaction area above the receptor, and the housing has an air outlet at one side of the water outlet, and the water outlet is connected to the air outlet in the housing.

Accordingly, another aspect of the present invention provides a detection method according to the chip-based cartridge, the detection method comprises the following steps: sample loading, washing, and detection. In the sample loading step, the sample is dropped directly into the reaction area and reacts with the receptor for a reaction time; in the washing step, after the reaction time, the wash buffer is dropped into the sample in the same reaction area such that the total volume of liquid exceeds that of the reaction area and the solution can come into contact with the microfluidic channel where it is then drawn in via capillary action, thus clearing the reaction area; in the detection step, after washing, detection buffer is dropped into the reaction area to perform sample detection.

In one embodiment, there is provided a detection method where in the sample loading step, the sample at the reaction area has a volume of 10˜15 μL, and the reaction time is 1˜5 minutes.

In one embodiment, there is provided a detection method, where in the washing step, a first drop of wash buffer should be dropped into the reaction area such that the total fluid volume is enough to be drawn into the microfluidic channel by capillary action. Next, a second drop of wash buffer is again dropped into the reaction area in the same way, again causing fluid to be drawn into the microfluidic channel by capillary action.

In one embodiment, there is provided a detection method, where in the detection step, a first drop of detection buffer should be dropped into the reaction area to allow the residue of the wash buffer and the detection buffer to be drawn into the microfluidic channel through capillary action, followed by a second drop of the detection buffer at the reaction area, wherein the second drop of the detection buffer stays in the reaction area to perform a sample detection.

Accordingly, another aspect of the present invention provides a detection method according to the chip-based cartridge, the detection method comprises the following steps: sample loading, washing, and detection. In the sample loading step, the sample is dropped directly into the reaction area and reacts with the receptor for a reaction time; in the washing step, after the reaction time, the wash buffer is dropped into the sample in the same reaction area such that the total volume of liquid exceeds that of the reaction area and the solution can come into contact with the microfluidic channel where it is then drawn in via capillary action, thus clearing the reaction area; in the detection step, after washing, detection buffer is dropped into the reaction area to perform sample detection; wherein the sample and the wash buffer or the detection buffer are absorbed by the adsorption part and discharged through the microfluidic channel quickly.

Accordingly, another aspect of the present invention provides a detection method according to the chip-based cartridge, the detection method comprises the following steps: sample loading, washing, and detection. In the sample loading step, the sample is dropped directly into the reaction area and reacts with the receptor for a reaction time; in the washing step, after the reaction time, the wash buffer is dropped into the sample in the same reaction area such that the total volume of liquid exceeds that of the reaction area and the solution can come into contact with the microfluidic channel where it is then drawn in via capillary action, thus clearing the reaction area; in the detection step, after washing, detection buffer is dropped into the reaction area to perform sample detection; wherein the size of the air outlet is adjusted to control the flow rate of the sample and the wash buffer or the detection buffer discharged through the microfluidic channel.

Therefore, the chip-based cartridge and the detection method thereof in the present invention allow the sample to be dropped directly into the reaction area and react immediately with the receptor. After the reaction, the sample and the wash buffer or detection buffer are drawn into the microfluidic channel and are discharged. In addition to completing the detection without external driving force, the sample can directly react with the receptor when dropped at the reaction area to shorten the detection time and improve the detection efficiency.

### BRIEF DESCRIPTION OF THE DRAWINGS

FIG. 1 is a schematic three-dimensional diagram of the chip-based cartridge for biological detection according to one embodiment of the present invention.

FIG. 2 is a schematic three-dimensional diagram of the body according to one embodiment of the present invention.

FIG. 3 is an exploded view of the body according to one embodiment of the present invention.

FIG. 4 is a top and exploded view of the body according to one embodiment of the present invention.

FIG. 5 is an exploded view of the housing according to one embodiment of the present invention.

FIG. 6 is a partial cross-sectional view of the chip-based cartridge for biological detection according to one embodiment of the present invention.

FIG. 7 is a flowchart of a detection method according to one embodiment of the present invention.

FIG. 8A is a schematic diagram of the chip-based cartridge for biological detection according to one embodiment of the present invention, illustrating dropping the sample at the reaction area.

FIG. 8B is a schematic diagram, continuing FIG. 8A, illustrating dropping the wash buffer (or the detection buffer) at the reaction area after the reaction time to infiltrate into the microfluidic channel and being discharged.

FIG. 8C is a schematic diagram, continuing FIG. 8B, illustrating dropping the detection buffer at the reaction area for the second time after the washing step.

FIG. 8D is a schematic diagram of the chip-based cartridge for biological detection according to one embodiment of the present invention, illustrating narrowing the air outlet of the housing.

### DETAILED DESCRIPTION OF THE INVENTION

In the following description, it will be apparent to those skilled in the art that modifications, including additions and/or substitutions may be made without departing from the scope and the spirit of the invention. Specific details may be omitted so as not to obscure the invention; however, the disclosure is written to enable one skilled in the art to practice the teachings herein without undue experimentation.

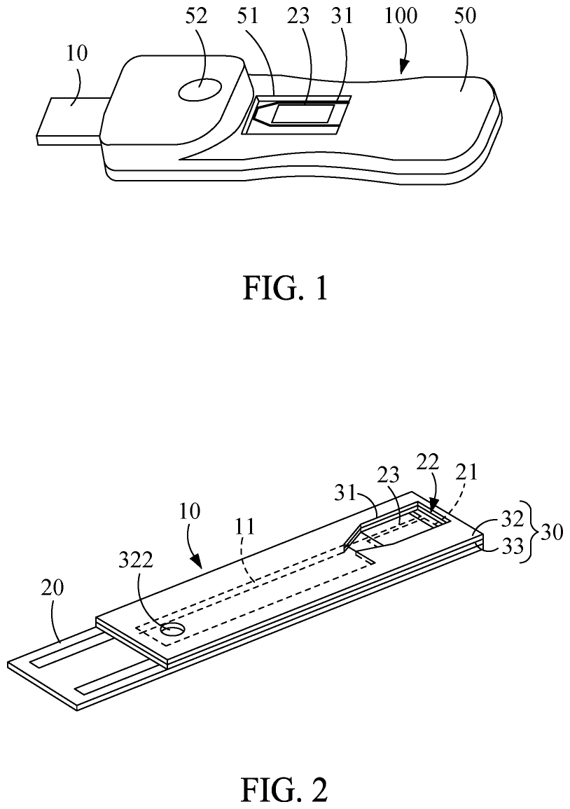

Referring to FIGS. 1 to 8D, the present invention provides a chip-based cartridge for biological detection 100 and a detection method 200 thereof. As shown in FIG. 1 to FIG. 6, the chip-based cartridge for biological detection 100 includes a body 10 having a substrate 20 and a flow guiding layer 30, and further includes an adsorption part 40 and a housing 50 in one embodiment, wherein:

The substrate 20 is provided with electrodes 21, and there is a reaction area 22 at the part of the pair of electrodes 21. The reaction area 22 is coated with a receptor 23 for binding a detection target. The detection target of the receptor 23 is, for example, but not limited to antibody, antigen, nucleic acid, or small molecule.

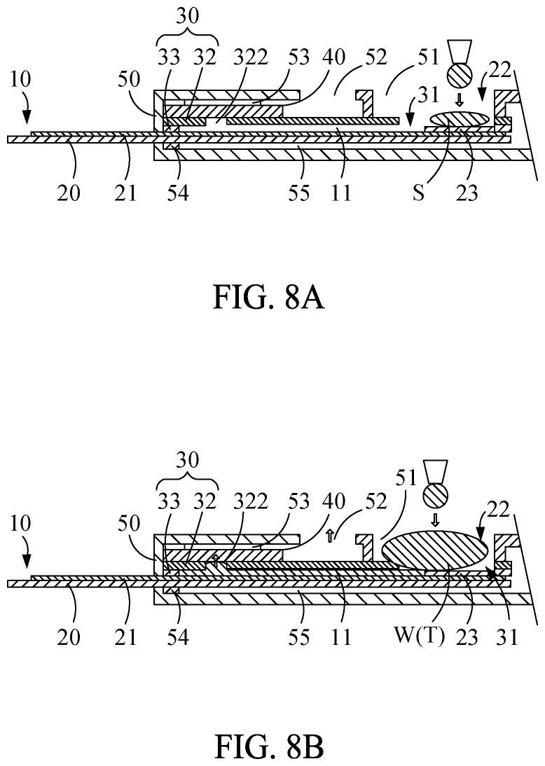

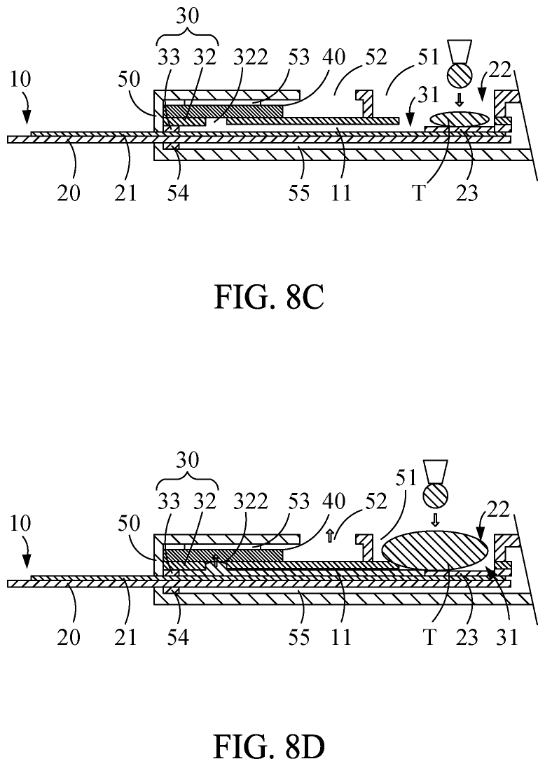

The flow guiding layer 30 is stacked with the substrate 20 to form the body 10. The flow guiding layer 30 has an opening 31, which is arranged to align with the reaction area 22 on site. Meanwhile, the receptor 23 is exposed at the opening 31 corresponding to the body 10, such that a sample S can be dropped directly at the reaction area 22 and react with the receptor 23 (as shown in FIG. 8A). The flow guiding layer 30 is provided with a microfluidic channel 11 between the flow guiding layer 30 and the substrate 20, and the microfluidic channel 11 is connected to the reaction area 22. The sample S is, for example, but not limited to blood.

As shown in FIG. 2 to FIG. 4, the flow guiding layer 30 includes a hydrophilic layer 32 and a hydrophobic layer 33, where the hydrophobic layer 33 is positioned between the substrate 20 and the hydrophilic layer 32 in the embodiment. The hydrophilic layer 32 is able to perform capillary action to absorb water, and the hydrophobic layer 33 does not absorb water. The opening 31 penetrates through the hydrophilic layer 32 and the hydrophobic layer 33 to the reaction area 22. The hydrophobic layer 33 has a space to form the microfluidic channel 11 between the substrate 20 and the hydrophilic layer 32.

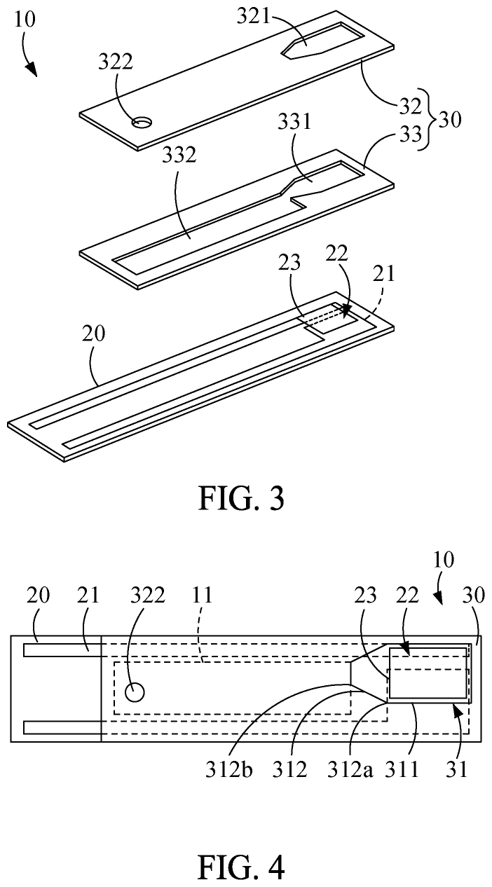

As shown in FIG. 3, the hydrophilic layer 32 in the embodiment has a first perforation 321, and the hydrophilic layer 32 has a water outlet 322 at one end of the microfluidic channel 11 away from the reaction area 22, while the hydrophobic layer 33 has a second perforation 331, and the first perforation 321 and the second perforation 331 coincide to form the opening 31; In addition, the hydrophobic layer 33 has a third perforation 332 to form the space, and the second perforation 331 and the water outlet 322 are communicated with both ends of the third perforation 332.

As shown in FIG. 4, the opening 31 is hexagonal, which has a reaction section 311 and a buffering section 312. The width of the reaction section 311 is equal, and the buffering section 312 forms a tapered shape. The buffering section 312 has a wide end 312a and a narrow end 312b. The wide end 312a is configured at one end of the reaction section 311, and the narrow end 312b communicates with the third perforation 332 of the hydrophobic layer 33 through the microfluidic channel 11. In the embodiment, the receptor 23 is disposed on the reaction section 311, and the buffering section 312 is configured to be as a buffer and regulate the flow rate. The shape of the opening 31 is for example, but not limited to the hexagonal in the present invention. With the hexagonal shape of the opening 31 and passing through the reaction area 22, the hydrophilic layer 32 does not perform capillary action in the opening 31. Moreover, the sample S is hindered by the hexagonal structure of the opening 31, such that the sample S, the wash buffer W or the detection buffer T will not overflow while dropping at the reaction area 22.

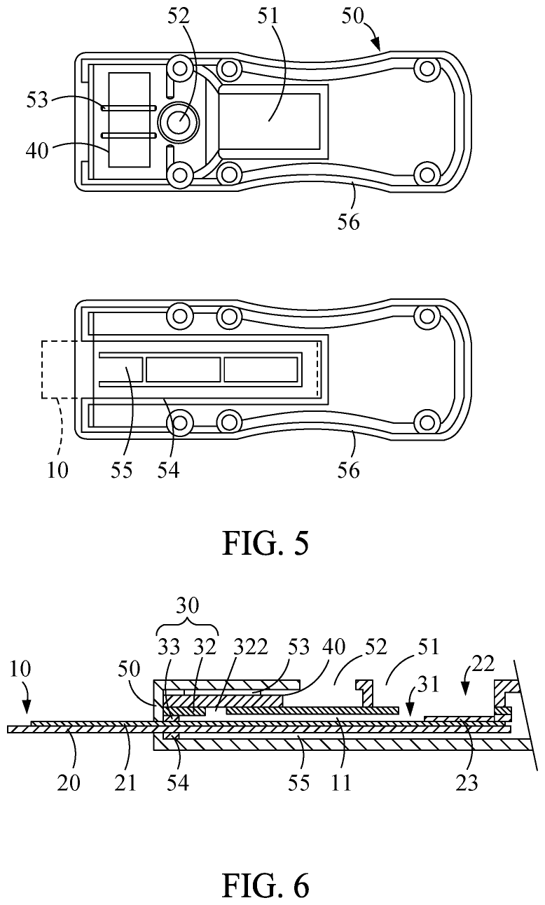

As shown in FIGS. 5 to 6, the body 10 and the adsorption part 40 are covered and positioned by the housing 50. The housing 50 has a window 51 passing through the opening 31 and passing through the reaction area 22 above the receptor 23, and the housing 50 has an air outlet 52 on one side of the water outlet 322. The water outlet 322 is connected to the air outlet 52 in the housing 50. In one embodiment, the adsorption part 40 is at the water outlet 322 configured on the hydrophilic layer 32.

As shown in FIGS. 5 to 6, the housing 50 has a fixing part 53 corresponding to the adsorption part 40, such that the fixing part 53 holds the adsorption part 40 at the water outlet 322 in order to position the adsorption part 40 without incurring any displacement. In the embodiment, the fixing part 53 are two ribs protruding from the housing 50, but the fixing part 53 in the present invention is not limited to as ribs. For example, the fixing part 53 can also be one or three more ribs, and the shape of the ribs includes but not limited to a linear shape, such as a round shape or a sheet shape. In other words, those that can achieve the function of holding adsorption part 40 at the water outlet 322 is subject to the protection scope of the fixing part 53 in the present invention; In addition, the housing 50 has a slot 54 corresponding to the body 10 for inserting the body 10, such that the reaction area 22 is aligned to the window 51. Moreover, the housing 50 has a rib 55 in the slot 54, and the body 10 at the slot 54 is hold and positioned by the rib 55. In the embodiment, the housing 50 includes two half housing parts 56.

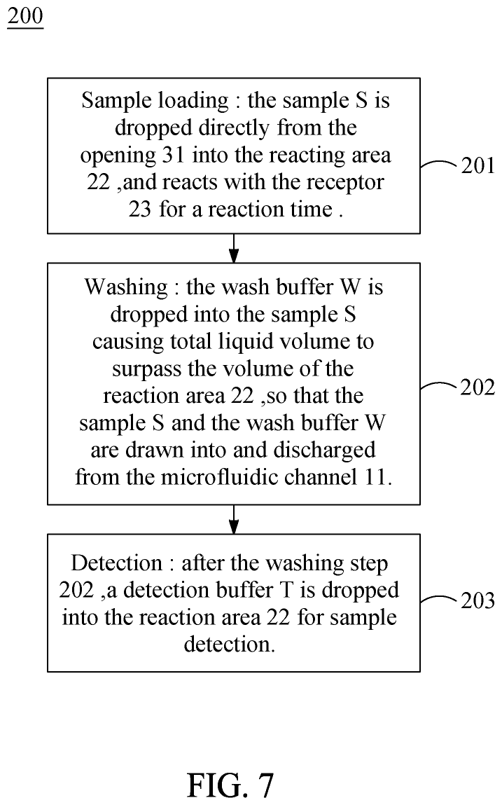

As shown in FIG. 7, the detection method 200 includes the steps of sample loading 201, washing 202 and detection 203, wherein:

In the sample loading steps 201, the sample S is dropped directly from the opening 31 into the reaction area 22 (as shown in FIG. 8A), and reacts with the receptor 23 for a reaction time. In one embodiment, the sample S performs the reaction in the reaction area 22 in a volume of 10˜15 μL, and the reaction time is 1˜5 minutes. The receptor 23 of the present invention includes but is not limited to immunoglobulins, nucleic acid probes, chemical molecules or functional proteins; the sample of the invention includes a detection target which can be bound to the receptor. In one embodiment, the receptor 23 is an IgG antibody or an IgM antibody, and the detection target in the sample S is virus or microorganisms that can specifically bind to the IgG antibody or IgM antibody.

In one embodiment, the receptor 23 is immunoglobulin, which is referred to as anti-COVID-19 RBD antibody (RBD antibody) with a concentration of 1-10 ug/mL. The detection target in the sample S is the COVID-19 receptor binding site protein (COVID-19 RBD) in saliva or nasal mucus, which can be detected from 1˜1000 μg/mL.

In the washing step 202, after the reaction time, the wash buffer W is dropped into the sample S causing total liquid volume to surpass the volume of the reaction area 22, so that the sample S and the wash buffer W are drawn into and discharged from the microfluidic channel 11 through capillary action provided by the hydrophilic layer 32 in the embodiment. In one embodiment, in the washing step 202 (as shown in FIG. 8B), the first drop of 35 μL wash buffer W is dropped to wash the sample S in the reaction area 22 so as to remove the non-specific binding substances to the receptor 23 in the sample S, and the sample S and wash buffer W are drawn into the microfluidic channel 11 to complete the first washing. Then, a second drop of 35 μL wash buffer is dropped in the reaction area 22, to wash the sample S to remove non-specific binding substances to the receptor 23 in the sample S again, and then the sample S and wash buffer W are drawn into the microfluidic channel 11 to complete the second washing. Therefore, the residues of the non-specific binding substances of the sample S in the reaction area 22 will not affect the test results.

In the detection step 203, after the washing step 202, a detection buffer T is dropped into the reaction area 22 for sample detection. In one embodiment, in the detection step 203 in the embodiment, the first drop of 35 μL detection buffer T is dropped into the reaction area 22, and the detection buffer T is able to infiltrate into the microfluidic channel 11 through capillary action (as shown in FIG. 8B), then the second drop of detection buffer T is dropped into the reaction area 22, and the second drop of detection buffer T stays in the reaction area 22 (as shown in FIG. 8C) to detect the sample reacted with the receptor 23 through the electrochemical reaction.

In the embodiment, both the sample S and wash buffer W are drawn into the microfluidic channel 11 through capillary action in the washing step 202, and the detection buffer T that is drawn into the microfluidic channel 11 through capillary action in the detection step 203 fills the microfluidic channel 11 and is discharged from the water outlet 322, upon which it is quickly absorbed by the adsorption part 40, such that the sample S and the wash buffer W in the washing step 202 or the detection buffer T in the detection step 203 can accelerate to be discharged from the microfluidic channel 11. The adsorption part 40 is a preferred embodiment in the present invention but not limited to this. That is, the sample S, the wash buffer W or the detection buffer T filling and flowing through the microfluidic channel 11 can also be naturally discharged from the water outlet 322. The difference is that the sample S, the wash buffer W or the detection buffer T can be quickly absorbed and accelerate to be discharged from the microfluidic channel 11 by the adsorption part 40.

In another embodiment, the size of the air outlet 52 is adjusted to control the flow rate of the sample S and the wash buffer W or the detection buffer T discharged from the microfluidic channel 11. As shown in FIG. 8A to FIG. 8C, when the air outlet 52 of the housing 50 is in large size, the sample S, the wash buffer W or the detection buffer T are discharged from the microfluidic channel 11 faster due to the large amount of exhaust capacity. In addition, as shown in FIG. 8D, when the air outlet 52 of the housing 50 is in small size, the sample S, the wash buffer W or the detection buffer T are discharged from the microfluidic channel 11 slowly due to the small amount of exhaust capacity. The size of the air outlet 52 is adjusted according to the demand to the flow rate of the sample S, the wash buffer W or the detection buffer T discharged from the microfluidic channel 11. In the embodiment, the air outlet 52 is in fixed size in the housing 50, and the size of the air outlet 52 is adjustable through the replacement of the housing 50. However, the present invention is not limited to this; for example, a flow valve (not shown in the figure) is configured to the housing 50, where the air outlet 52 is formed in the flow valve, and the size can be adjusted by the flow valve, such that the flow rate of the sample S, the wash buffer W or the detection buffer T discharged from the microfluidic channel 11 can be controlled.

According to the above description, the unique features in the present invention are as follows:

(1) The chip-based cartridge for biological detection 100 and the detection method 200 of the present invention, where the sample S can be directly dropped into the reaction area 22 and the sample S and the receptor 23 can react first, then the sample S and the wash buffer W are drawn into the microfluidic channel 11 and discharged. In contrast, with the conventional detection device, the sample must first be drained to the reaction area through the capillary action and then reacted with receptor afterwards. In addition to completing the sample detection without external driving force, the present invention can directly react with the receptor 23 when the sample S is dropped into the reaction area 22, so as to avoid the delay of the detection time caused by the sample draining to the reaction area, leading to shorter detection time and improve the detection efficiency.

(2) The opening 31 is hexagonal and the opening 31 is directly connected to the reaction area 22, therefore the hydrophilic layer 32 does not perform capillary action in the opening 31. The opening 31 with hexagonal structure can form a barrier for the sample S, such that the sample S, the wash buffer W or the detection buffer T dropped into the reaction area 22 will not overflow, resulting in a smooth detection process and improved the detection efficiency.

(3) With the adsorption part 40, the sample S, the wash buffer W or the detection buffer T are quickly absorbed by the adsorption part 40 when they are discharged from the microfluidic channel 11. Compared with the speed of natural discharge, this design can accelerate the process to improve the detection efficiency.

The invention has been disclosed above with preferred embodiments, for those familiar with the art should understand that the embodiment is only used to describe the invention and should not be interpreted as limiting the scope of the invention. It should be noted that all changes and substitutions equivalent to the embodiment should be included in the scope of the present invention. Therefore, the scope of protection of the invention shall be subject to the scope of the patent application.

1. A chip-based cartridge for biological detection comprising a body, the body comprising:
a substrate having electrodes including a reaction area positioned on the part of the electrodes, the reaction area coated with a receptor for binding a detection target; and
a flow guiding layer stacked with the substrate, the flow guiding layer having an opening aligned with the reaction area;
wherein the receptor is exposed at the opening corresponding to the body such that a sample can be dropped directly into the reaction area and immediately react with the receptor;
wherein a microfluidic channel is formed between the flow guiding layer and the substrate, connecting to the reaction area;
wherein the sample after the reaction is discharged from the microfluidic channel by adding wash buffer such that both sample and wash buffer are drawn into the microfluidic channel via capillary action.

2. The chip-based cartridge of claim 1, wherein the flow guiding layer comprises a capillary hydrophilic layer and a non-absorbent hydrophobic layer, the hydrophobic layer is positioned between the substrate and the hydrophilic layer, the opening penetrates through the hydrophilic layer and the hydrophobic layer to the reaction area, and the hydrophobic layer has a space to form the microfluidic channel between the substrate and the hydrophilic layer.

3. The chip-based cartridge of claim 2, wherein the opening is hexagonal and has a reaction section with equal width and a buffering section with tapered shape, the buffering section has a wide end and a narrow end, the wide end is arranged at one end of the reaction section, and the narrow end is connected to the space of the hydrophobic layer to connect to the microfluidic channel.

4. The chip-based cartridge of claim 2, wherein the chip-based cartridge further comprises an adsorption part, the hydrophilic layer has a water outlet at one end of the microfluidic channel away from the reaction area, and the adsorption part is positioned on the water outlet configured to the hydrophilic layer.

5. The chip-based cartridge of claim 4, wherein the chip-based cartridge further comprises a housing, the body and the adsorption part are covered and positioned by the housing, the housing has a window passing through the opening to the reaction area above the receptor, and the housing has an air outlet at one side of the water outlet, and the water outlet is connected to the air outlet in the housing.

6. The chip-based cartridge of claim 1, wherein the receptor is selected from immunoglobulins, nucleic acid probes, chemical molecules, or functional proteins.

7. A detection method according to the chip-based cartridge of claim 1, comprising steps of:
sample loading: loading the sample directly into the reaction area from the opening and allowing the sample to react with the receptor for a reaction time;
washing: after the reaction time, dropping the wash buffer onto the sample at the reaction area to surpass the volume of the reaction area, such that the sample and the wash buffer are drawn into the microfluidic channel through capillary action and are discharged from the channel; and
detection: after washing, dropping detection buffer into the reaction area to perform sample detection.

8. The detection method of claim 7, wherein the sample loading step, the sample at the reaction area has a volume of 10˜15 μL, and the reaction time is 1˜5 minutes.

9. The detection method of claim 8, wherein the washing step, the washing further comprises a first washing and a second washing; wherein the first washing comprises dropping a first drop of the wash buffer at the reaction area to wash the sample such that the sample and the wash buffer can be drawn into the microfluidic channel to complete the first washing; wherein the second washing comprises dropping a second drop of the wash buffer at the reaction area to wash the sample such that the sample and the wash buffer are drawn into the microfluidic channel to complete the second washing.

10. The detection method of claim 8, wherein the detection step, the detection further comprises dropping a first drop of detection buffer at the reaction area to allow the residue of the wash buffer and the detection buffer to be drawn into the microfluidic channel through capillary action, followed by dropping a second drop of detection buffer at the reaction area, wherein the second drop of detection buffer stays in the reaction area to perform the sample detection.

11. A detection method according to the chip-based cartridge of claim 4, comprising steps of:
sample loading: loading the sample directly into the reaction area from the opening and allowing the sample to react with the receptor for a reaction time;
washing: after the reaction time, dropping the wash buffer onto the sample at the reaction area to surpass the volume of the reaction area, such that the sample and the wash buffer are drawn into the microfluidic channel through capillary action and are discharged from the channel; and
detection: after washing, dropping detection buffer into the reaction area to perform sample detection;
wherein the sample and the wash buffer or the detection buffer are absorbed by the adsorption part and discharged through the microfluidic channel quickly.

12. The detection method of claim 11, wherein the sample loading step, the sample at the reaction area has a volume of 10˜15 μL, and the reaction time is 1˜5 minutes.

13. The detection method of claim 12, wherein the washing step, the washing further comprises a first washing and a second washing; wherein the first washing comprises dropping a first drop of the wash buffer at the reaction area to wash the sample such that the sample and the wash buffer can be drawn into the microfluidic channel to complete the first washing; wherein the second washing comprises dropping a second drop of the wash buffer at the reaction area to wash the sample such that the sample and the wash buffer are drawn into the microfluidic channel to complete the second washing.

14. The detection method of claim 12, wherein the detection step, the detection further comprises dropping a first drop of detection buffer at the reaction area to allow the residue of the wash buffer and the detection buffer to be drawn into the microfluidic channel through capillary action, followed by dropping a second drop of detection buffer at the reaction area, wherein the second drop of detection buffer stays in the reaction area to perform the sample detection.

15. A detection method according to the chip-based cartridge of claim 5, comprising steps of:
sample loading: loading the sample directly into the reaction area from the opening and allowing the sample to react with the receptor for a reaction time;
washing: after the reaction time, dropping the wash buffer onto the sample at the reaction area to surpass the volume of the reaction area, such that the sample and the wash buffer are drawn into the microfluidic channel through capillary action and are discharged from the channel; and
detection: after washing, dropping detection buffer into the reaction area to perform sample detection;
wherein the size of the air outlet is adjusted to control the flow rate of the sample and the wash buffer or the detection buffer discharged through the microfluidic channel.

16. The detection method of claim 15, wherein the sample loading step, the sample at the reaction area has a volume of 10˜15 μL, and the reaction time is 1˜5 minutes.

17. The detection method of claim 16, wherein the washing step, the washing further comprises a first washing and a second washing; wherein the first washing comprises dropping a first drop of the wash buffer at the reaction area to wash the sample such that the sample and the wash buffer can be drawn into the microfluidic channel to complete the first washing; wherein the second washing comprises dropping a second drop of the wash buffer at the reaction area to wash the sample such that the sample and the wash buffer are drawn into the microfluidic channel to complete the second washing.

18. The detection method of claim 16, wherein the detection step, the detection further comprises dropping a first drop of detection buffer at the reaction area to allow the residue of the wash buffer and the detection buffer to be drawn into the microfluidic channel through capillary action, followed by dropping a second drop of detection buffer at the reaction area, wherein the second drop of detection buffer stays in the reaction area to perform the sample detection.
